(19) United States
(12) Patent Application Publication
(10) Pub. No.: US 2025/0363630 A1
(43) Pub. Date: Nov. 27, 2025

(54) COMPUTER-IMPLEMENTED METHOD FOR PROCESSING A MODEL OF A VESSEL STRUCTURE OF A PATIENT, PROCESSING DEVICE, COMPUTER PROGRAM AND ELECTRONICALLY READABLE STORAGE MEDIUM

(71) Applicant: Siemens Healthineers AG, Forchheim (DE)

(72) Inventors: Michael WELS, Bamberg (DE); Christian HOPFGARTNER, Fuerth (DE); Felix LADES, Erlangen (DE); Michael SUEHLING, Erlangen (DE)

(73) Assignee: Siemens Healthineers AG, Forchheim (DE)

(21) Appl. No.: 19/214,632

(22) Filed: May 21, 2025

(30) Foreign Application Priority Data
May 22, 2024 (EP) 24177469.4

(51) Int. Cl. G06T 7/00; A61B 6/50; G06T 7/62; G06T 19/20; G16H 50/50

(52) U.S. Cl. CPC G06T 7/0012; G06T 7/62; G06T 19/20; G16H 50/50; A61B 6/504; G06T 2200/04; G06T 2200/24; G06T 2207/30101; G06T 2210/41; G06T 2219/2021

(57) ABSTRACT

A method for processing a three-dimensional model of a vessel structure comprises: providing the model, the model including a topological graph representation of the vessel structure and first courses of centerlines of vessels; receiving user input data describing at least one corrected course of a centerline; determining, from the user input data and the model, an affected section of the chosen centerline, at least one non-affected section of the chosen centerline, and, for each branch-off along the affected section, a modifiable section connected to the affected section, and a non-modifiable section outside the affected section; determining a deformation field from the affected section, the non-affected section, the non-modifiable section, and the corrected course, such that the deformation field maps the first course of the chosen centerline to the corrected course without changing non-modifiable sections; and applying the deformation field to the affected section and all modifiable sections.

### CROSS-REFERENCE TO RELATED APPLICATION(S)

The present application claims priority under 35 U.S.C. § 119 to European Patent Application No. 24177469.4, filed May 22, 2024, the entire contents of which are incorporated herein by reference.

### FIELD

One or more example embodiments of the present invention concern a computer-implemented method for processing a model of a vessel structure of a patient, a processing device, a computer program and a non-transitory electrically readable storage medium. Features and advantages described in connection with the method according to one or more example embodiments of the present invention can also be implemented as corresponding features, units, or modules in the processing device and in the computer program, and vice versa.

### BACKGROUND

In medical imaging, methods are known to acquire image data relating to vessel structures in a patient, for example blood vessel structures and in particular blood vessel trees, like the coronary artery tree. Three-dimensional patient images of vessel structures can be acquired, for example, using magnetic resonance imaging or computed tomography (CT), in particular computed tomography angiography (CTA). In particular, contrast agents may be used to provide improved contrast. Well-known algorithms, in particular analysis functions trained by machine learning, are used to derive geometrical, three-dimensional models of the vessel structure. In a common approach, centerlines of vessels may be automatically determined and/or vessel lumen segmentation may be performed. Exemplarily regarding centerline determination, it is referred to Y. Zheng et al., “Robust and Accurate Coronary Artery Centerline Extraction in CTA by Combining Model-Driven and Data-Driven Approaches”, in: In International Conference on Medical Image Computing and Computer-Assisted Intervention, Nagoya, Japan, MICCAI '13, pages 74-81, September 2013, or to Y. Zheng et al, “Model-driven centerline extraction for severely occluded major coronary arteries”, in: Machine Learning in Medical Imaging, LNCS 7588, pages 10-18, 2012.

Automatically determined models may, however, still have inaccuracies. It was therefore proposed to provide users with the possibility to edit the model, in particular in a visualization of the model in the underlying three-dimensional patient image. Often, the views in which, for example, centerlines or boundaries may be edited by a user, are multiplanar reformations (MPRs) or curved planar reformations (CPRs). The CPRs may be defined to follow the course of the centerlines of a certain vessel. Such a method for correcting centerlines (and also vessel lumen segmentations) is, for example, described in M. Wels et al., “Intuitive and accurate patient-specific coronary tree modeling from cardiac computed-tomography angiography”, in: Interactive Medical Image Computing (Int. Conf. Med. Image Comput. Comput.-Assist. Interv. 2016 Workshop Proceedings), Athens, GR, pages 86-93, October 2016.

U.S. Pat. No. 10,733,787 B2 discloses a method for interactively generating a geometric model of a vessel structure on the basis of three-dimensional image data of an examination region of interest of a patient. A representation of the vessel structure is determined on the basis of three-dimensional image data and a two-dimensional representation is determined on the basis of the determined representation using a preferably non-linear planar reformation of the three-dimensional vessel structure. Subsequently, boundary indicators which define the surface profile of the volume object are edited in the two-dimensional representation. Following the editing, a three-dimensional representation of the edited boundary indicators is generated by back-transforming the edited boundary indicators into three-dimensional space.

Regarding the three-dimensional model of the vessel structure, it has been proposed to model the vessel tree as a bundle of independent centerlines having common parts. In an improved approach, topological graphs, in particular trees, were used to describe the vessel structure in three-dimensional models. Such data structures are known from computer technology. In concrete embodiments, it was suggested to represent the entire geometric object as a proper computer-scientific tree having nodes with sequences of 3D points, which describe centerlines. Each node hence contains the course of the centerline of the respective vessel leading from the parent node to the respective current node, which can also be stored in other descriptions, for example as a function instead of a sequence of points. Each tree node further comprises pointers to its child nodes, which in turn describe the child vessel courses to these child nodes, allowing to model complex vascularization as a recursive hierarchical data structure. An editing and processing approach adapted for three-dimensional models using such topological graphs was described in a publication by M. Wels et al., “Consistent Hierarchical 3D Vessel Tree Editing”, in: PLEASE ADD CITATION HERE! The publication describes seven use cases for editing of a model of a vessel structure.

However, this and related approaches suffer from drawbacks. Editing operations across bifurcations could change the tree topology in an undesired and clinically implausible manner. Parts of the previous vessel course tend to persist in the tree structure as additional vessels. This may even not be noticed by the user, as the superficial impression on the view, in which the editing is done, in particular a CPR view, which does not show the rest of the tree, suggests successful editing. Furthermore, non-connected vessel crossings may be introduced.

### SUMMARY

It is an object of one or more embodiment of the present invention to provide an improved processing approach based on user input data describing a course change of a centerline, which respects and preserves the underlying true physical topology of the vessel structure.

At least this object is achieved by providing a computer-implemented method, a processing device, a computer program and an electronically readable storage medium according to the independent claims. Advantageous embodiments are described by the dependent claims.

A method for processing a three-dimensional model of a vessel structure of a patient, according to an embodiment of the present invention, comprises the steps of
providing the model of the vessel structure, the model comprising a topological graph representation of the vessel structure and first courses of centerlines of the vessels,
receiving user input data describing at least one corrected course of a chosen centerline,
determining, from the user input data and the model,
an affected section of the chosen centerline affected by the correction, and
at least one non-affected section of the chosen centerline outside the affected section, and, for each branch-off, in particular bifurcation, along the affected section
a modifiable section of the respective branch-off centerline, wherein the modifiable section is connected to the affected section, and
a non-modifiable section of the respective branch-off centerline outside the affected section,
determining a deformation field from the affected section, the non-affected section, the non-modifiable section, and the corrected course, such that the deformation field maps the first course of the chosen centerline to the corrected course of the chosen centerline and does not change any non-modifiable sections,
applying the deformation field to the affected section and all modifiable sections to determine updated courses to be stored in the model as new first courses.

In an additional, optional step, the stored model with the new first courses can be outputted as a data set to a respective interface. The stored model may also be referred to as an “updated model”. In this way, the stored model can be outputted, e.g. to one or more of a data connection, a data storage, data network, and a display device. Additionally or alternatively, a graphic representation of an updated three-dimensional model of the vessel structure, which is based on the stored model (or updated model), can be outputted, e.g. to one or more of a data connection, a data storage, data network, and a display device.

While this method may be applied to any vessel structure, which can be described as a (computer-scientific) topological graph, preferably, the vessel structure may be a blood vessel structure (vascular structure), in particular a coronary vessel tree. In particular regarding such a coronary vessel tree, but also in other applications (for example for blood vessel structures in the brain or other organs), the vessel structure may be described in the model as a tree, which will be often used as an illustrating example here. In other words, three-dimensional vessel trees can be represented in a model as (computer-scientific) trees as topological graphs. Information regarding the vessels (for example their boundaries and, in particular, the centerline courses), may be stored in edges and/or, preferably, in nodes. In preferred embodiments, in each node of the tree, the first course of the vessel segment leading from a previous node to the respective node and subsequent nodes may be stored.

Preferably, the model may be determined by evaluating a three-dimensional patient image of the vessel structure, in particular applying at least one trained evaluation function to the image data. In particular, the patient image may comprise a magnetic resonance image and/or a computed tomography image. Preferably, the patient image is an angiographic image. For example, an approach as described by Y. Zheng et al. in “Model-driven centerline extraction for severely occluded major coronary arteries”, Machine Learning in Medical Imaging, LNCS 7588, Pages 10-18, 2012, or other known approaches may be used to extract centerlines and/or vessel boundaries/lumens from the patient image. The three-dimensional model may then be constructed from these determined vessel properties, but may, as already discussed, suffer from inaccuracies. Hence, the user is provided the option to correct the model, in particular regarding the courses of the centerlines.

For example, the user corrected course may be defined with respect to a planar reformatted image of a three-dimensional patient image showing the vessel structure, in particular a curved planar reformation (CPR) following the course of the chosen vessel (the vessel along the chosen centerline). Herein, the patient image is registered to the model, as will be the case anyways if the model is derived from the patient image. The user input data may be the result of an editing operation, in particular created by interaction with the depiction of one individual centerline—the chosen centerline—either on an MPR or a CPR view. Known techniques described in the above-mentioned publications by M. Wels et al. may also be applied here.

One or more embodiments of the present invention provide a topology-preserving modification of the three-dimensional model based on user input data describing a corrected course of a chosen centerline. The steps of the method apply to continuous, that is non-interrupted, sections of the chosen centerline, while, of course, multiple such corrections may each be treated on their own, for example, successively. Hence, the affected section and any modifiable sections are understood to be continuous, i.e., not interrupted.

Preferably, the method described here is applied to corrections along the course of a centerline. That is, the steps of determining the deformation field as well as the step of applying the deformation field may be only performed if the affected section is located between non-affected sections. Other editing cases, for example corrected courses ending at a distance to an existing centerline, adding a new centerline to the vessel structure, or deleting a centerline shall not be treated using this new approach, since existing approaches already preserve the topology in such cases. For example, steps as described in “Consistent Hierarchical 3D Vessel Tree Editing” by M. Wels, as cited above, may be used.

It is noted that one or more embodiments of the present invention are particularly advantageous whenever branch-offs are affected, which is the non-trivial case in centerline correction, since it concerns multiple nodes and/or edges in the topological graph. Hence, preferably, the steps of determining the deformation field as well as the step of applying the deformation field may at least be performed if the affected section passes a branch-off, leading to the definition of a non-zero number of modifiable sections.

In embodiments of the present invention, it is proposed to determine a deformation field based on the corrected course in the affected section (known from the user input data), which can also be applied to the modifiable section, where the correction is not known from the user input data. To provide a proper definition of the deformation field, it does not change the course of the centerlines in the non-affected sections and the non-modifiable sections. In other words, the deformation field is hence determined not to modify the non-affected section and the non-modifiable section. The deformation field may then also be applied to the modifiable section, resulting in a smooth and consistent editing operation.

Advantageously, by only modifying the course in centerline sections which are already present in the topological graph, the overall graph topology is preserved during editing. Hence, the physical correlations describing the real-world vessel structure are upheld without producing any non-physical artifacts. The method described here is less prone to degeneration of the whole geometric vessel structure as editing and graph update are better encapsulated. The editing operation is not applied directly, but an intermediate deformation field is estimated, which is then applied to the graph. The steps described can, in particular, be implemented in real time, allowing smooth editing even of more complex vessel trees.

The edited/corrected three-dimensional model can be used in many applications. Geometrically modeling vessel trees is an important prerequisite when it comes to further analysis of vessel structures depicted in medical patient images, e.g., with regards to atherosclerosis or simulated fluid dynamics. Easy interactive modeling capabilities that help the user to model vessel geometry exactly to his needs is crucial for the acceptance of such analysis tools. Embodiments of the present invention allow manual overriding of results generated by evaluation functions, in particular artificial intelligence (AI), while preserving the correct physical basis. Furthermore, interactive tools for acquiring ground-truth annotations for training and evaluating machine learning-based automatic evaluation solutions for vessel tracing and the like may also use the technique described here.

In concrete embodiments, the affected section is determined by
running along the chosen centerline and determining, in particular orthogonal, distances between the first course and corrected course;
defining the affected section as all parts of the centerline, where the distance is larger than an affection threshold.

Usually, the affection threshold may be very small, such that all editing operations of the user are taken into account. For example, the threshold may correspond to a smallest measurable distance, for example a voxel size.

Preferably, all modifiable sections are determined by calculating a distance measure to the affected section along the vessel structure, that is, first courses, and selecting parts having distance measure up to distance threshold. Hence, with knowledge about the affected part, other parts of the vessel tree may be assigned a distance measure to the affected section, i.e. the region where editing took place. Generally speaking, the distance threshold may chosen dependent on the size of the chosen vessel, in particular a diameter, and/or a strength of correction, in particular a maximum distance determined to define the affected section. For example, the distance threshold may be defined to comprise one to four times the diameter of the chosen vessel in the affected section and/or one to four times the maximum distance determined to define the affected section. It is important to emphasize that the distance measures are, of course, determined along the first courses, since non-connected vessel parts are, of course, less or even not affected by any correction (in the sense of a “pulling” or “pushing” of the chosen vessel). In concrete embodiments, a Dijkstra algorithm may be used to compute shortest distances in the topological graph.

In preferred embodiments, generally, the centerlines and the corrected course may be defined by representative centerline points, wherein the sections are defined as sets of centerline points. The distance of the points along the centerlines may, for example, follow a predefined definition scheme. The points may be uniformly distributed along the centerlines, in particular having the same distance along the centerlines, but may also be otherwise distributed, in particular according to curvature of the centerline, according to an underlying grid, and the like. If, as discussed above, in each node of the tree, the first course of the vessel segment leading from a previous node (parent node) to the respective node and subsequent nodes (child nodes) are stored, the points are stored in the nodes in this case. For example, in the case of a tree, each tree node may hold a set of centerline points, which can be understood as a 3D polygon and describes the first course of a respective vessel segment, with arbitrary and not necessarily uniform sampling. Each tree node further stores pointers to its child nodes, hence, for example, modeling vascularization as a hierarchical data structure.

Using points to describe the courses of centerlines has the advantage that only certain sampling positions have to be used to determine the deformation field, allowing robust and fast determination. Furthermore, regarding sets of points, in particular three-dimensional polygons, approaches for determining deformation fields have already been proposed in the state of the art for other applications. These approaches, accordingly modified, can also be applied in the present method.

When determining the deformation field and using points, the non-affected and non-modifiable sections can easily be modelled. Centerline points in the non-affected section and the non-modifiable section are simply considered stationary, modelling a constraint.

In concrete, advantageous embodiments, intermediate points on the affected section may be defined as positions of orthogonal distance from the centerline points of the corrected course to the affected section, wherein the three-dimensional deformation field is determined based on pairs of corrected course centerline points and respective intermediate points to describe the correction in the affected section. In other words, orthogonal projections of the intermediate points onto the corrected course yield the centerline points of the corrected course. In the non-affected section and in the non-modifiable section, further pairs may be defined by the stationary centerline points.

In any case, pairs of centerline points result, from which the deformation field can be easily estimated. In other words, the point pairs serve as samples for the continuous three-dimensional deformation field.

Here, in especially preferred embodiments, the deformation field may be interpolated using radial basis functions. From the point pairs and the radial basis functions, a linear system of equations is established, which may be solved using fast numerical methods, in particular solving functions. Preferably, least-squares solvers and/or single value decomposition (SVD) can be used. The point pairs can be reduced to a certain interpolation region around the affected section, such that stationary points which are further away can be excluded from the computation. This also prevents the introduction of numerical variations as these centerline points are supposed to remain at their original location anyways.

In an article by M. Botsch and L. Kobbelt, “Real-time shape editing using radial basis functions”, in: Computer Graphics Forum, 24, 2005, pages 611-621, a related topology-preserving mesh surface editing strategy is proposed, the basic principles of which can also be applied in the currently described method. Mesh surface points are, however, less sensitive to where exactly on the surface they end up after editing, as this will not alter the surface itself. In embodiments of the present invention, the expanded semantics of vessel centerline points, in particular in coronary tree structures, is additionally taken into account by applying constraints, such that the global influence of local editing operations on the overall shape can be restricted. These constraints comprise the centerline points in the non-affected and non-modifiable sections being stationary. In this manner, editing one chosen centerline cannot alter the course of a distant vessel.

Hence, generally speaking, the deformation field may be determined by interpolation using radial basis functions. Once the deformation field has been determined, it is applied to the affected section and the modifiable sections, in particular their centerline points. This may lead to deviations from a point distribution scheme, if used.

Hence, in preferred embodiments, at least for the affected section and all modifiable sections, the centerline points of the updated courses may be adapted according to a point distribution scheme, in particular uniform point distribution. In this optional step, the resulting centerline points can be resampled to match the original sampling convention, that is, point distribution scheme, of the topological graph. In particular, a uniform point distribution may be required for analysis and/or postprocessing functions to be applied to the edited model of the vessel structure. However, in preferred embodiments, the adaptation may alternatively or additionally comprise deleting at least one centerline point where a centerline point density along the updated course is higher than a predetermined density defined by the point distribution scheme. In this manner, “squeezed” distributions, in particular in modifiable sections, may be resolved.

Embodiments of the present invention also concern a processing device, comprising at least one processor and at least one storage device and/or means, wherein the processing device further comprises, for processing a three-dimensional model of a vessel structure of a patient:
a first interface for receiving the model of the vessel structure, the model comprising a topological graph representation of the vessel structure and first courses of centerlines of the vessels,
a second interface for receiving user input data describing at least one corrected course of a chosen centerline,
a first determination unit for determining, from the user input data and the model,
an affected section of the chosen centerline affected by the correction, and
at least one non-affected section of the chosen centerline outside the affected section, and, for each branch-off, in particular bifurcation, along the affected section,
a modifiable section of the respective branch-off centerline, wherein the modifiable section is connected to the affected section, and
a non-modifiable section of the respective branch-off centerline outside the affected section,
a second determination unit for determining a deformation field from the affected section, the non-affected section, the non-modifiable section, and the corrected course, such that the deformation field maps the first course of the chosen centerline to the corrected course of the chosen centerline and does not change any non-modifiable sections,
an updating unit for applying the deformation field to the affected section and all modifiable sections to determine updated courses to be stored in the model as new first courses.

The processing device may further comprise a third interface for outputting the updated model. In this way, the stored or updated model can be outputted, e.g. to one or more of a data connection, a data storage, data network, and a display device. Additionally, or alternatively, a graphic representation of an updated three-dimensional model of the vessel structure, which is based on the stored model (or updated model), can be outputted, e.g. to one or more of a data connection, a data storage, data network, and a display device.

All features and remarks regarding the method according to embodiments of the present invention may also be applied to the processing device according to embodiments of the present invention and vice versa, such that the same advantages can be achieved.

In particular, the processing device may be part of a control device of a medical imaging device, for example an x-ray device and/or a magnetic resonance device, and/or a workstation. In particular, at least one input device and at least one output device may be associated to the processing device. Using the output device, visualizations of the model, in particular the mentioned MPRs and/or CPRs, may be output, comprising respective image data as a basis for assessment by a user. User input data may be generated by the input device, in particular when the user interacts with the visualization to perform an editing operation on the chosen centerline.

A computer program, according to embodiments of the present invention, comprises a computer program and/or program means such that, when the computer program is executed on a processing device, the processing device is caused to perform the steps of a method according to embodiments of the present invention. The computer program may be stored on an electronically readable storage medium according to embodiments of the present invention, which thus has control information stored thereon, the control information comprising at least one computer program according to embodiments of the present invention, such that, when the storage medium is used in a processing device, the processing device is configured to perform a method according to embodiments of the present invention. The storage medium according to embodiments of the present invention may be non-transient or non-transitory, for example a CD-ROM.

### BRIEF DESCRIPTION OF THE DRAWINGS

Other objects and features of the present invention will become apparent from the following detailed description considered in conjunction with the accompanying drawings. The drawings, however, are only principal sketches designed solely for the purpose of illustration and do not limit the present invention. The drawings show:

FIG. 1 a flowchart of an embodiment of a method according to embodiments of the present invention,

FIG. 2 a first schematical illustration showing a tree segment of a topological tree structure of a vessel structure and a user-defined corrected course of a chosen centerline,

FIG. 3 a second schematical illustration showing distances from an affected section and corresponding definitions,

FIG. 4 a third schematical illustration showing the definition of centerline point pairs as samples for determining a deformation field,

FIG. 5 a fourth schematical illustration showing the tree segment deformed by the deformation field,

FIG. 6 a fifth schematical illustration showing resampled centerline points, and

FIG. 7 the functional structure of a processing device according to embodiments of the present invention.

### DETAILED DESCRIPTION

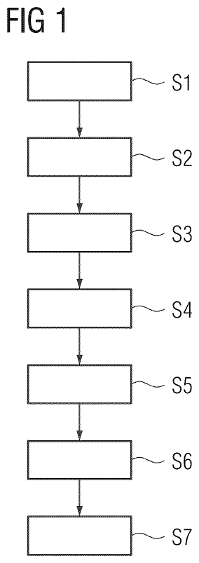

FIG. 1 is a flowchart of an embodiment of a method, according to embodiments of the present invention, for processing a three-dimensional model of a vessel structure of a patient, for example a coronary artery tree. The method may be embedded into an editing module, where a user can perform editing operations on at least the centerlines of the model. The method described here handles editing operations that change the first course of a chosen centerline to a corrected course in an affected section between two non-affected sections of the chosen centerline and is at least applied if there is at least one bifurcation in the affected section.

In a step S1, the three-dimensional model is received via a first interface. In a step S2, user input data describing the corrected course is received as result of an editing operation via a second interface. The model has been determined by evaluating a three-dimensional patient image of the vessel structure using a trained evaluation function. The patient image may comprise a magnetic resonance image and/or a computed tomography image. Centerlines and/or vessel boundaries/lumens have been extracted from the patient image and the three-dimensional model was constructed from these determined vessel properties. Here, since a vessel tree is concerned as the vessel structure, the model comprises a tree representation of the vessel structure. The tree is an example of a topological graph. In this embodiment, each node of the tree stores the first course of the vessel segment leading from a previous node (parent node) to the respective node and subsequent nodes (child nodes). The first courses are stored as sets of three-dimensional centerline points, which may follow a certain point distribution scheme. In the embodiment presented here, uniform point distribution is used, but other schemes may also be employed.

Since the automatically determined model may suffer from inaccuracies, the user is provided the option to correct the model, in particular regarding the courses of the centerlines. Here, the corrected course may be defined by interacting with a planar reformatted image of the three-dimensional patient image, in particular a curved planar reformation (CPR) following the course of the chosen vessel.

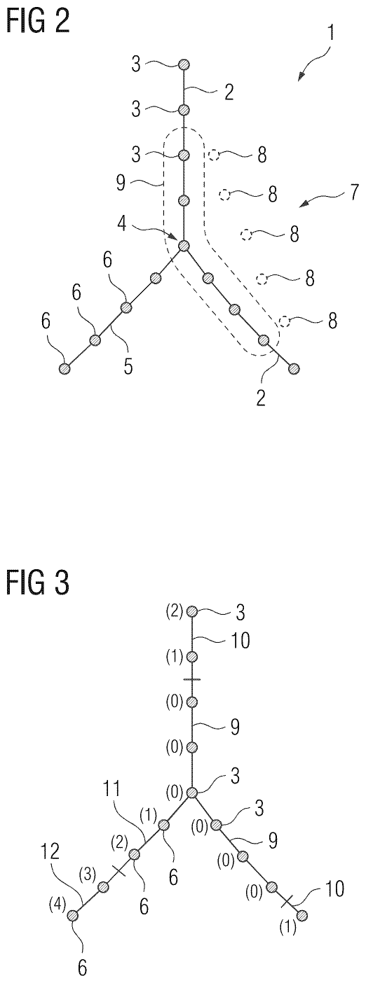

FIG. 2 schematically shows a tree segment 1 of the modelled vessel structure. It is noted that the simplified view is provided for better explanation of the applied processing method. In reality, vessel structures are more complex regarding size and degree of ramification.

As can be seen, the chosen centerline 2 is defined by multiple centerline points 3. At a bifurcation 4, a branch-off centerline 5 with respective centerline points 6 leads away from the chosen centerline 2. Hence, a node is defined at the bifurcation 4. FIG. 2 also shows the user corrected course 7, which also has centerline points 8. The corrected course 7 spans the bifurcation 4.

Returning to FIG. 1, in a step S3, certain sections are determined using the model and the user input data. First of all, an affected section 9 already indicated in FIG. 1 is determined as the section of the chosen centerline 2 where the centerpoints 8 of the corrected course 7 deviate from the centerline points 3 of the first course of the chosen centerline 2, in particular by more than an affection threshold. All other centerline points 3 of the chosen centerline 2 belong to non-affected sections 10 (see FIG. 3).

For all branch-off centerlines 5, distance measures along the first courses in the vessel structure to the affected sections 9 are now determined using a Dijkstra algorithm. In the example presented here, due to the uniform distribution, the distance measures can be expressed exemplarily as integers, shown in FIG. 3 in parentheses. All centerline points 6 along the branch-off centerline 5, whose distance measure is smaller than a distance threshold, in this case three, are added to a modifiable section 11, which, consequently, is connected to the affected section, as indicated in FIG. 3. All other centerline points 6 of the branch-off centerline 5 belong to a non-modifiable section 12.

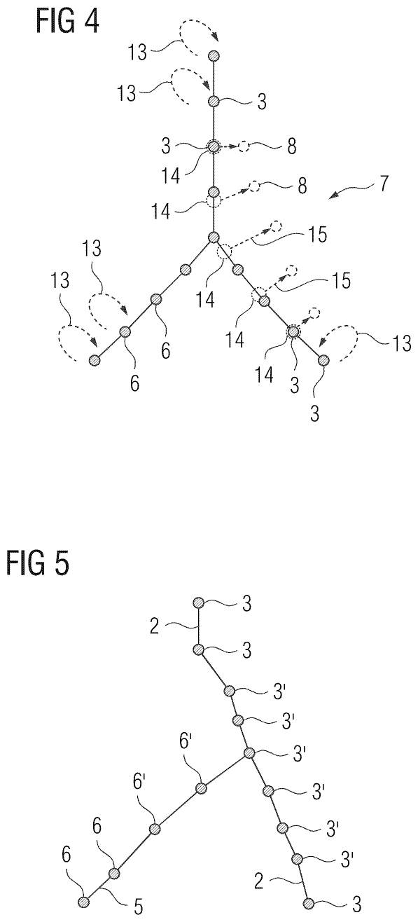

In a step S4 of FIG. 1, a deformation field is then determined. In a first substep, centerline point pairs are defined, which serve as a basis for interpolating the deformation field. As the centerline points 3 and 6 of the non-affected and non-modifiable sections 10, 12, respectively, are supposed to be stationary, as indicated by arrows 13 in FIG. 4, centerline point pairs can easily be generated with themselves. These pairs serve as constraints. In the affected section 9, however, intermediate points 14 (dotted) are defined as positions of orthogonal distance from the centerline points 8 of the corrected course 7 to the affected section 9. This can be understood as a resampling of the affected section 9 to match the sampling of the edited, corrected course 7. Pairs of corrected course centerline points 8 and respective intermediate points 14 are formed to describe the correction in the affected section 9 (indicated by arrows 15) and serve as samples for the three-dimensional deformation field.

In a second substep of step S4, the deformation field is determined by interpolation using radial basis functions. A system of linear equations is defined and solved using a solving function, for example employing SVD.

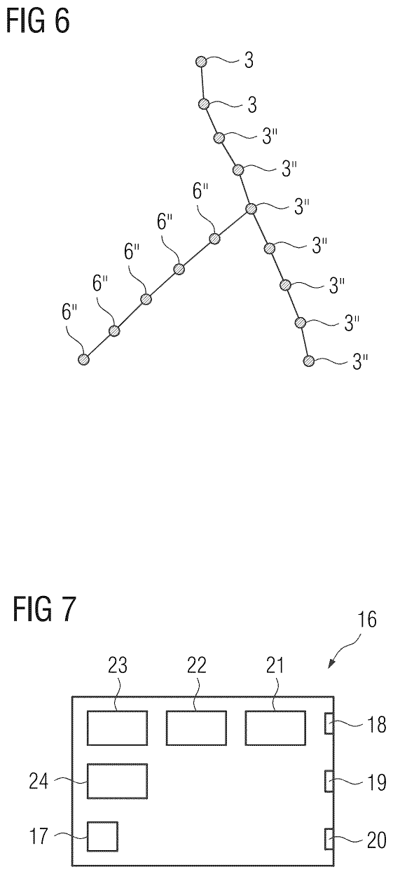

In a step S5, the determined deformation field is then applied to the affected section 9 and the modifiable section 11, resulting in new centerline points 3′ and 6′, respectively, as shown in FIG. 5. As can be seen, these centerline points 3′ and 6′ are not according to the uniform point distribution scheme, such that, in an optional step S6, they may be adapted to uniformly distributed centerline points 3″ and 6″ as shown in FIG. 6. The step S6 may also, independently from the uniform point distribution scheme, serve to delete centerline points 3′, 6′ if they are to close due to “squeezing” by the deformation field.

In a step S7, the updated courses are stored as new first courses in the model and the updated model is output via a third interface.

Additionally in step S7, the stored or updated model can be outputted, e.g. to one or more of a data connection, a data storage, data network, and a display device. Additionally, or alternatively, a graphic representation of an updated three-dimensional model of the vessel structure, which is based on the stored model (or updated model), can be outputted, e.g. to one or more of a data connection, a data storage, data network, and a display device.

FIG. 7 shows a functional drawing of a processing device 16. The processing device, which may be part of a control device of a medical imaging device or of a workstation, comprises a storage device and/or storage means 17, a first interface 18 for receiving the model in step S1, a second interface 19 for receiving the user input data in step S2 and a third interface 20 for outputting the updated model in step S7. In a first determination unit 21, the affected section 9, the non-affected sections 10, the modifiable sections 11 and the non-modifiable sections 12 are determined according to step S3. The processing device further comprises a second determination unit 22 for determining the deformation field according to step S4. In an updating unit 23, the deformation field is applied to the model according to step S5. In an optional adaptation unit 24, the centerline point distribution may be adapted according to step S6.

Independent of the grammatical term usage, individuals with male, female or other gender identities are included within the term “patient”.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for processing a three-dimensional model of a vessel structure of a patient, the computer-implemented method comprising:
providing the three-dimensional model of the vessel structure, the three-dimensional model including a topological graph representation of the vessel structure and first courses of centerlines of vessels of the vessel structure;
receiving user input data describing at least one corrected course of a chosen centerline;
determining, from the user input data and the three-dimensional model,
an affected section of the chosen centerline affected by the at least one corrected course,
at least one non-affected section of the chosen centerline outside the affected section, and
for each respective branch-off along the affected section,
a modifiable section of a respective branch-off centerline, wherein the modifiable section is connected to the affected section, and
a non-modifiable section of the respective branch-off centerline outside the affected section;
determining a deformation field from the affected section, the at least one non-affected section, the non-modifiable section, and the at least one corrected course, such that the deformation field maps a first course of the chosen centerline to the at least one corrected course of the chosen centerline and does not change any non-modifiable sections; and
applying the deformation field to the affected section and all modifiable sections to determine updated courses to be stored in the three-dimensional model as new first courses.

2. The computer-implemented method according to claim 1, wherein the affected section is determined by
running along the chosen centerline and determining distances between the first course and the at least one corrected course, and
defining the affected section as all parts of the chosen centerline, where a distance is larger than an affection threshold.

3. The computer-implemented method according to claim 1, wherein all modifiable sections are determined by
calculating a distance measure to the affected section along the vessel structure, and
selecting parts having a distance measure up to a distance threshold.

4. The computer-implemented method according to claim 3, wherein the distance threshold is chosen depending on at least one of a size of a vessel of the chosen centerline or a strength of correction.

5. The computer-implemented method according to claim 1, wherein the centerlines and the at least one corrected course are defined by representative centerline points, wherein sections are defined as sets of centerline points.

6. The computer-implemented method according to claim 5, wherein intermediate points on the affected section are defined as positions of orthogonal distance from the representative centerline points of the at least one corrected course to the affected section, wherein the deformation field is determined based on pairs of corrected course centerline points and respective intermediate points to describe a correction in the affected section.

7. The computer-implemented method according to claim 5, wherein, at least for the affected section and all modifiable sections, the representative centerline points of the updated courses are adapted according to a point distribution scheme.

8. The computer-implemented method according to claim 7, wherein adaptation of the representative centerline points comprises:
deleting at least one centerline point where a centerline point density along an updated course is higher than a density defined by the point distribution scheme.

9. The computer-implemented method according to claim 1, wherein the deformation field is determined by interpolation using radial basis functions.

10. The computer-implemented method according to claim 1, wherein the vessel structure is described in the three-dimensional model as a tree.

11. The computer-implemented method according to claim 1, wherein the determining the deformation field and the applying the deformation field are only performed if at least one of (i) the affected section passes a branch-off or (ii) the affected section is located between non-affected sections.

12. The computer-implemented method according to claim 1, wherein the three-dimensional model is determined by evaluating a three-dimensional patient image of the vessel structure.

13. A processing device, comprising:
at least one storage device; and
at least one processing device configured to process a three-dimensional model of a vessel structure of a patient, the at least one processing device including
a first interface configured to receive the three-dimensional model of the vessel structure, the three-dimensional model including a topological graph representation of the vessel structure and first courses of centerlines of vessels of the vessel structure,
a second interface configured to receive user input data describing at least one corrected course of a chosen centerline,
a first determination unit configured to determine, from the user input data and the three-dimensional model,
an affected section of the chosen centerline affected by the at least one corrected course,
at least one non-affected section of the chosen centerline outside the affected section, and
for each respective branch-off along the affected section
a modifiable section of a respective branch-off centerline, wherein the modifiable section is connected to the affected section, and
a non-modifiable section of the respective branch-off centerline outside the affected section,
a second determination unit configured to determine a deformation field from the affected section, the at least one non-affected section, the non-modifiable section, and the at least one corrected course, such that the deformation field maps a first course of the chosen centerline to the at least one corrected course of the chosen centerline and does not change any non-modifiable sections, and
an updating unit configured to apply the deformation field to the affected section and all modifiable sections to determine updated courses to be stored in the three-dimensional model as new first courses.

14. A non-transitory computer-readable medium storing computer-executable instructions that, when executed at at least one processing device, cause the at least one processing device to perform the computer-implemented method of claim 1.

15. The computer-implemented method of claim 1, wherein each respective branch-off along the affected section is a bifurcation along the affected section.

16. The computer-implemented method of claim 2, wherein the distances between the first course and the at least one corrected course are orthogonal distances.

17. The computer-implemented method of claim 4, wherein at least one of
the chosen centerline is a diameter, or
the strength of correction is a maximum distance determined to define the affected section.

18. The computer-implemented method of claim 10, wherein each node of the tree stores a first course of a vessel segment leading from a previous node to a respective node and subsequent nodes.

19. The computer-implemented method according to claim 12, wherein the evaluating the three-dimensional patient image of the vessel structure includes applying at least one trained evaluation function to image data of the three-dimensional patient image.

20. The processing device of claim 13, wherein each respective branch-off along the affected section is a bifurcation along the affected section.

21. The computer-implemented method according to claim 7, wherein the point distribution scheme is a uniform point distribution.
